SEQUENCE LISTING (9 sequences)

SEQ ID NO: 1 - protein, 14 aa, Homo sapiens
LEEKKGNYVVTDHC

SEQ ID NO: 2 - protein, 6 aa, Homo sapiens
LEEKKC

SEQ ID NO: 3 - protein, 8 aa, Homo sapiens
NYVVTDHC

SEQ ID NO: 4 - protein, 5 aa, Homo sapiens
VLSNY

SEQ ID NO: 5 - protein, 8 aa, Homo sapiens
EKKGNYVV

SEQ ID NO: 6 - protein, 3 aa, Homo sapiens
KGN

SEQ ID NO: 7 - protein, 3 aa, Homo sapiens
EKK

SEQ ID NO: 8 - protein, 7 aa, Homo sapiens
NYVVTDH

SEQ ID NO: 9 - protein, 3 aa, Homo sapiens
NYV

(19) United States
(12) Patent Application Publication
(10) Pub. No.: US 2001/0046686 A1
(43) Pub. Date: Nov. 29, 2001

(54) Sensitive detection of wild-type and mutant EGFR by specific ELISA assays in any biological sample

(76) Inventors: Albert J. Wong, Philadelphia, PA (US); Kim E. Leitzel, Hummelstown, PA (US); David K. Moscatello, Philadelphia, PA (US); Allan Lipton, Hershey, PA (US)

Correspondence Address: THOMAS JEFFERSON UNIVERSITY, INTELLECTUAL PROPERTY DIVISION, 1020 WALNUT STREET, SUITE 620, PHILADELPHIA, PA, 19107, US

(21) Appl. No.: 09/803,854

(22) Filed: Mar. 12, 2001

Related U.S. Application Data
(60) Provisional application No. 60/188,424, filed on Mar. 10, 2000.

(51) Int. Cl. G01N 33/574; G01N 33/537

(52) U.S. Cl. 435/7.23; 435/7.92

(57) ABSTRACT

The present invention generally relates a method of detecting type III mutant EGF receptor (EGFRvIII) in biological samples, a method of detecting cancers and other diseases in biological samples, and to a method of assessing treatment and selecting therapy for cancer patients.

### CROSS REFERENCE TO RELATED APPLICATIONS

This application claims priority under 35 U.S.C. § 119 based upon U.S. Provisional Patent Application No. 60/188,424 filed Mar. 10, 2000.

### GOVERNMENT RIGHTS IN THE INVENTION

This invention was made with government support under grants 51093 and 69495 awarded by the National Institutes of Health. The government has certain rights in the invention.

### FIELD OF THE INVENTION

The present invention generally relates to the fields of immunology and medicine and to a method of diagnosing cancers and other diseases in biological samples and, more particularly, to a method of detecting type III mutant EGF receptor (EGFRvIII) in biological samples, a method of detecting cancers and other diseases in biological samples, and a method of assessing treatment and selecting therapy for cancer patients.

### BACKGROUND OF THE INVENTION

The success of any cancer therapy is based upon its ability to distinguish neoplastic cells from normal cells. Most current chemotherapy or radiotherapy regimens are based upon differential growth rates of tumor cells. In practice, such therapies have been very successful in treating some cancers, but for many other cancers current treatments are either palliative in nature or in the long term are ineffectual. Progress in brain tumor therapy has been especially poor as the survival curve has not appreciably changed in over 60 years. Some progress has been made using biologically based modalities such as harvesting a patient's immune system or therapeutics based upon recent research in molecular biology. However, the specificity of these therapeutics for cancerous cells is poor. Much of the research in biology based therapies has focused on defining tumor specific alterations.

Detection of mutant and wild type growth factors, oncogenes, and tumor markers has played a critical role for detection and response to therapy in many diseases. For example, in oncology the detection of CEA (carcinoembryonic antigen) and PSA (prostate specific antigen) have played a major role in cancer diagnostics (1). More recently, the HER2neu/c-erb2 oncogene has played a critical role in cancer progression and response to therapy (2, 3, 4).

A related growth factor receptor, the epidermal growth factor receptor (EGFR) is an 170 kD membrane-spanning receptor that regulates differentiation and growth in both normal and neoplastic cells. Elevated levels of EGFR have been reported in many human tumors and cell lines, including breast cancer, adenocarcinoma and squamous lung cancer, gastrointestinal cancers (gastric, colon, pancreatic), renal cell cancer, bladder cancer, glioma, gynecological carcinomas, and prostate cancer.

Witters, Lipton, and colleagues have recently reported that the ectodomain of EGRF can be detected in the urine of 15 of 42 (36%) squamous cell carcinoma patients, 8 of 50 (16%) of patients with non-squamous carcinoma, and only 3 of 50 (6%) healthy control individuals (5). The presence of EGFR ectodomain in the urine of the patients with squamous cell carcinoma also correlated with the stage of disease, since 10 of 19 (53%) of patients with metastatic disease had elevated urine EGFR, compared to 5 of 23 (22%) of patients with localized disease. Others have reported the presence of increased ectodomain of EGFR in the serum of asbestosis patients (6), however Witters and Lipton were unable to report a difference in serum EGFR between cancer patients and healthy control individuals (Witters and Lipton, unpublished observation).

The type III mutant EGF receptor (EGFRvIII) results from an in-frame deletion from joining nucleotides 274 to 1076 in the EGFR cDNA sequence creating a new epitope at the fusion junction. This in-frame deletion corresponds to a deletion of amino acids 6 to 273 in the extracellular region, which causes constitutive activation of the tyrosine kinase domain. This variant or mutation occurs frequently in ovarian, breast, lung and glioblastoma cancers but has not been reported in normal tissues. Using a polyclonal anti-EGFRvIII-specific antibody, Moscatello, Wong, and colleagues have detected this mutant protein in 16% of non-small cell lung tumors, 78% of breast carcinomas, 57% of primary human glial tumors, 86% of medulloblastoma tumors, and 75% of ovarian tumors (7-9). Furthermore, the EGFRvIII is tumor specific, since it is not detected in any normal tissue examined (7-10).

Because EGFRvIII is tumor specific, an assay which can detect and quantify EGFRvIII in urine, serum/plasma, CSF, amniotic fluid, breast secretions, lung sputum, and tumor cell extracts may be of critical importance in the early detection of various cancers, and also in prognosis, monitoring, and response to therapy. In addition, this assay could serve in the selection of cancer patients for novel mutant EGF-directed anticancer therapies, such as a vaccine (7), antibody-toxin conjugate (11), or EGFRvIII-specific tyrosine kinase inhibitors (12).

The present invention involves such an assay. In the present invention, an EGFRvIII-specific ELISA was developed using a combination of polyclonal and monoclonal antibodies directed against the deletion junction domain. In the present invention, an ELISA specific for wild-type EGFR only (not EGFRvIII) was also developed.

The preparation and use of antibodies against EGFRvIII as described by Bigner/Vogelstein U.S. Pat. Nos. 5,212,290; 5,401,828; 5,710,010; 5,814,317; and in Wikstrand et al. (Journal of Neuroimmunology, 46:165, 1993), and Humphrey et al. (Proc. Natl. Acad. Sci., 87:4207, 1990) does not yield a preparation that is specific for solely EGFRvIII (see FIG. 1, Moscatello et al., Cancer Res. 55:5536, 1997). Such small quantities of antibodies against the wild type EGF receptor are sufficient to produce erroneous data on the presence of EGFRvIII in both ELISA and immunohistochemistry.

By contrast in the present invention, a purification method is devised that will yield antibodies that strictly recognize EGFRvIII and do not show any cross reactivity with the wild type EGF receptor.

### SUMMARY OF THE INVENTION

These novel ELISA assays that discriminate for the first-time between mutant and wild type EGFR show strong potential for the early detection, prognosis, monitoring, and evaluation of response to therapy of patients with a variety of cancers and other pathologic conditions; and for the selection of cancer patients for novel mutant EGF-directed anticancer therapies such as a vaccine or antibody-toxin conjugate. These ELISAs could be used to detect mutant and/or wild type-specific EGFR in any biologic fluid, including but not limited to urine, serum/plasma, CSF, amniotic fluid, breast secretions, lung sputum, and tumor cell extracts.

The present invention is a method of detecting type III mutant EGF receptor (EGFRvIII) in biological samples, a method of detecting cancers and other diseases in biological samples, and a method of assessing treatment and selecting therapy for cancer patients.

### BRIEF DESCRIPTION OF THE FIGURE

FIG. 1: FIG. 1 demonstrates that the antibody is indeed specific for EGFRvIII. 50 μg of cell lysates from cells expressing EGFRvIII (HC2) or cells that express the wild type EGF receptor (A431), were run on SDS-PAGE and transferred to nitrocellulose membranes. These blots were then incubated with antibodies against EGFRvIII using the three affinity columns as described (anti-EGFRvIII), or an antibody against wild type EGF receptor (anti-wt EGFR). Note that the anti-EGFRvIII preparation only recognizes the EGFRvIII protein and not the wt EGF receptor despite the presence of comparable amounts of each protein in the cell lysates.

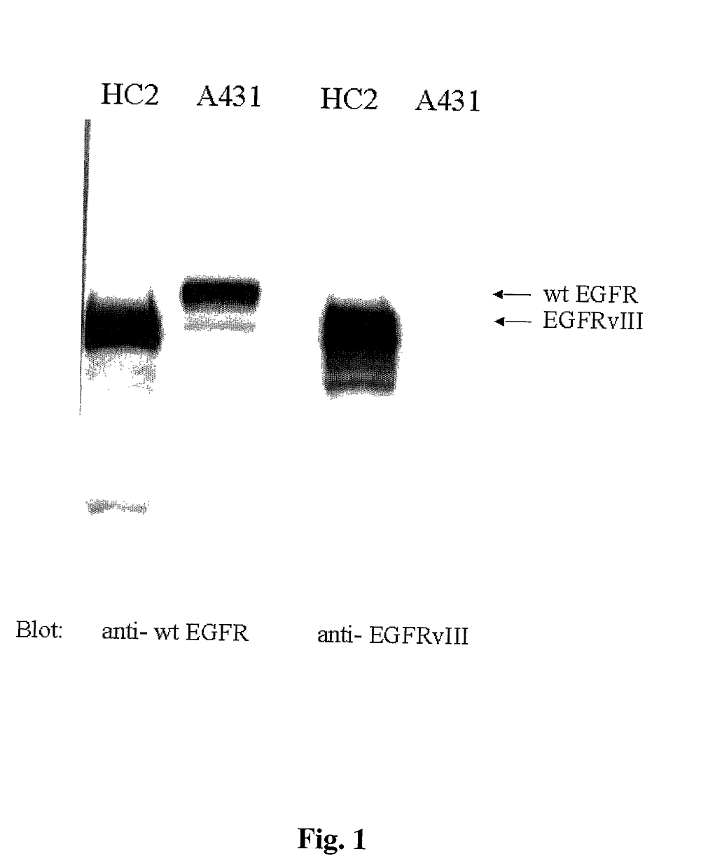

### DETAILED DESCRIPTION OF THE INVENTION

The present invention relates to the development of a purification method that yields antibodies that strictly recognize EGFRvIII and do not show any cross reactivity with wild type (wt) EGF receptor. Generally, the method of antibody preparation is a method of generating antibodies specific for EGFRvIII, comprising: preparation of an antibody against the mutant EGF receptor by immunizing a mammal with at least one of a mutant receptor protein, an epitope of said mutant receptor protein, a sequence that mimics said epitope, or DNA encoding said mutant receptor protein or epitope; obtaining a high titer antibody preparation from said mammal, said antibody preparation recognizing mutant EGF and wild type (wt) receptor; pooling bleeds from said mammal, concentrating and partially purifying said bleeds by precipitation; obtaining a pellet from said precipitation and dialyzing said pellet; and passing said dialyzed pellet over an affinity matrix column and eluting antibodies from said column to obtain antibodies specific for EGFRvIII. Alternatively, antibodies specific for EGFRvIII can be obtained by immunizing a mammal with at least one of a mutant receptor protein, an epitope of said mutant receptor protein, a sequence that mimics said epitope, or DNA encoding said mutant receptor protein or epitope; obtaining serum from said; and passing serum over an affinity matrix column and eluting antibodies from said column to obtain antibodies specific for EGFRvIII.

More specifically, in the preferred method, the antibody against the mutant EGF receptor was first prepared by immunizing New Zealand White rabbits with pepEGFRvIII (LEEKKGNYVVTDHC [SEQ ID NO:1]) conjugated to Keyhole Limpet Hemocyanin (KLH). The initial vaccination was 100 mg in complete Freund's adjuvant. Rabbits were subsequently boosted approximately every six weeks with KLH-pepEGFRvIII mixed with Freund's incomplete adjuvant, and rabbits were bled 7 to 10 days later. A high titer antibody preparation that recognized both EGFRvIII and wt EGF receptor was obtained after six to nine weeks. Sera were pooled from bleeds from weeks nine and later and then concentrated and partially purified by precipitation with 50% saturated ammonium sulfate. The pellet was dialyzed against several changes of PBS.

To obtain antibodies that were specific for EGFRvIII, this dialyzed material was passed over an affinity matrix column containing 2 mgs of pepEGFRvIII conjugated to 2 mls of Pierce Sulfo-Link Beads (Pierce Chemical Company, IL). Antibodies were eluted from this column using 50 mM glycine, pH 2.5. The resulting antibody eluates were then dialyzed against PBS.

Although the antibodies thus obtained recognized EGFRvIII, cross-reactivity with the normal EGFR was observed. To obtain antibodies solely specific for EGFRvIII, this antibody preparation was further purified by passing over an affinity matrix column to which was bound the peptide LEEKKC (SEQ ID NO:2), where the first five amino acids are derived from the normal EGF receptor sequence and the C-terminal cysteine was added for the purposes of conjugation to the Sulfo-link matrix. The flow through from this column was then passed over an affinity matrix column containing the peptide NYVVTDHC (SEQ ID NO:3), where the first seven amino acids are derived from the normal EGF receptor and the C-terminal cysteine is for conjugation purposes. The flow-through antibody recognized only EGFRvIII, whereas the antibodies, which bound to the LEEKKC (SEQ ID NO:2) and NYVVTDHC (SEQ ID NO:3) columns, cross-reacted with the normal EGFR. The novel, secondary affinity purification steps involving the use of the LEEKKC (SEQ ID NO:2) and NYVVTDHC (SEQ ID NO:3) columns were necessary to prepare antibody of specificity to be used in ELISA and immunohistochemistry protocols.

Thus, in the present invention, an EGFRvIII-specific ELISA was developed using a combination of polyclonal and monoclonal antibodies directed against the deletion junction domain. An extract of NIH-3T3 cells transfected with EGFRvIII (HC2 20d2/c cell line) was employed to generate a standard curve. No cross-reactivity was observed in the EGFRvIII ELISA when purified wild-type EGFR was tested.

In the present invention, an ELISA specific for wild-type EGFR only (not EGFRvIII) was also developed, and this ELISA detected no reactivity in the extracts of the HC2 20d2/c cell line. Sensitivity of the EGFRvIII ELISA was 6-10 ng/ml of HC2 20d2/c extract.

Steps for the developed ELISA systems are as follows:

1. The ELISA begins by coating the Immulon 4 ELISA wells with a polyclonal coating Ab. The coating Abs:

a. For EGFRvIII ELISA only—AP Anti-EGFRvIII—Supplied by Dave Moscatello and Albert Wong of Thomas Jefferson University. This polyclonal rabbit Ab is supplied as 1 μg/μl in PBS.

b. For wtEGFR ELISA only—Ab 1068—Supplied by Dave Moscatello and Albert Wong of Thomas Jefferson University. This polyclonal rabbit Ab is supplied as 1 μg/μl in PBS with 0.5 μg/μl BSA. Ab 1068 recognizes phosphorylated and nonphosphorylated EGFR, both wt and EGFRvIII.

2. Refrigerate overnight

3. Wash with PBS-Tween

4. Block ELISA plate for at least two hours with 300 μl/well of 1% Gelatin-PBS. The plate is incubated at room temperature with shaking.

5. Wash with PBS-Tween

6. Add Standards (Antigen)

a. HC2 Lysate—Supplied by Dave Moscatello and Albert Wong of Thomas Jefferson University. This is a cell extract that contains EGFRvIII (1.94 mg/ml)

b. WtEGFR—Supplied by Sigma (Catalog E-3641, Lot 128H4074) in a volume of 500 units (0.42 mg/ml) from human carcinoma A431 cells. This is a wild type EGFR standard.

c. A431 Lysate—Supplied by Dave Moscatello and Albert Wong of Thomas Jefferson University.

7. Refrigerate overnight

8. Wash with PBS-Tween

9. Add Secondary Ab

a. For EGFRvIII ELISA only—Ab 10—Monoclonal indicator Ab supplied by Neomarkers (MS-378-P1). Ab 10 can bind standards/antigen of both wtEGFR and EGFRvIII.

b. For wtEGFR ELISA only—Ab 16—Monoclonal indicator Ab supplied by Neomarkers (MS-666-PO). [Ab 16 shows good reactivity with wild type EGFR; however, Ab 16 is NOT reactive with EGFRvIII (unpublished observation, B. L. Marshall, K. Leitzel, A. Lipton)].

10. Incubate two hours at room temperature with shaking

11. Wash with PBS-Tween

12. Add Jackson Biotinylated Goat Anti-Mouse Conjugate (catalog 115-065-146)

a. Prepare a 1:25000 dilution and add 100μl to each well

13. Incubate 30 minutes at room temperature with shaking

14. Prepare Vectastain Elite ABC reagents supplied by Vector Laboratories and allow to sit for 30 minutes in the dark

15. Wash with PBS

16. Add Vectastain Elite ABC reagents supplied by Vector Laboratories in the amount of 100 μl/well.

17. Incubate 30 minutes at room temperature with shaking

18. Wash with PBS

19. Add 100 μl/well TMB Substrate supplied by Kiregaard & Perry Laboratories Inc. (Catalog 50-76-00)

a. While monitoring at 650 nm, wait until the well of highest reactivity reads 0.6 OD.

b. Add 100 μl/well of H₃PO₄ stop solution

c. Read plate at dual wavelength of 450-595 nm

The ELISA assays of the present invention are for the first time able to detect exclusively mutated EGFRvIII (EGFRvIII ELISA) and/or exclusively wild-type EGFR (wtEGFR ELISA). Currently available EGFR ELISA cannot discriminate between these two EGFR forms.

In experiments preformed with the mutant EGFRvIII ELISA (EGFRvIII ELISA), HC2 Lysate (EGFRvIII) was employed to demonstrate a typical standard curve for this ELISA.

Furthermore, a comparison of coating antibodies was performed to determine if cross-reactivity exists between these antibodies and mutant and wild type EGFR. The coating Abs compared were as follows:

1. Ab 1068 (immunooprecipitates both mutant and wild type EGFR)

2. Anti-VLSNY (SEQ ID NO:4) (binds wild type EGFR in Western blot but not by immunoprecipitation, therefore it is incapable of binding with the wild type EGFR in ELISA

3. “old” Anti-EGFRvIII (stock coating Ab from the summer of 1997) (recognizes mutant EGFRvIII)

4. “new” Anti-EGFRvIII (stock coating Ab received June 1999) (recognizes mutant EGFRvIII)

From the assay performed it was determined that when using wild type EGFR (Sigma) there was no reactivity in the wells coated with anti-EGFRvIII yet there was substantial activity in the wells coated with Ab 1068 (recognizes both mutant and wild type EGF) as expected. Therefore, in the EGFRvIII ELISA there is no cross-reactivity with wild type EGFR.

Detection in Urine

When our ELISA was used to detect EGFRvIII in clinical urine samples from cancer patients with known elevations of wild type EGFR from a previous study (4), up to a 3-fold elevation in reactivity was noted. Increasing the sensitivity of our assay yields an even greater frequency and magnitude of detection of mutant EGFRvIII.

Detection in Breast Primary Tumor Pellet Extracts

We have previously published the utility of the HER-2/neu ELISA to quantify HER-2/neu in breast cancer pellet extracts for predicting prognosis in breast cancer patients (13). When our EGFRvIII ELISA was used to detect EGFRvIII in primary tumor extract samples from breast cancer patients, up to a 2 log-fold range in reactivity was noted. Therefore, quantitative evaluation of EGFRvIII by our ELISA proves critical to determining the prognostic and response to therapy potential of EGFRvIII in any human cancer extracts.

### REFERENCES

1. Hayes, D F. Serum (circulating) tumor markers for breast cancer. Recent Results in Cancer Research. Vol 140: 101-113, 1996. Springer-Verlag Berlin Heidelberg Publishing.

2. Leitzel, K. E., Demers, L., Bartholomew, M., Harvey, H., and Lipton, A.: Elevated c-erbB-2 levels in the serum of a proportion of breast cancer patients. Breast Cancer Res. Treatment 16: 191, 1990.

3. Leitzel, K., Teramoto, Y., Sampson, E., Mauceri, J., Langton, B. C., Demers, L., Podczaski, E., Harvey, H., Shambaugh, S., Volas, G., Weaver, S., and Lipton, A.: Elevated soluble c-erbB-2 antigen levels in the serum and effusions of a proportion of breast cancer patients. J. Clin. Oncol., 10:1436-1443, 1992.

4. Leitzel K, Teramoto Y, Konrad K, Chinchilli V M, Volas G, Grossberg H, Harvey H, Demers L, Lipton A: Elevated serum c-erbB-2 antigen levels and decreased response to hormone therapy of breast cancer. J. of Clin. Oncol. 13:1129, 1995.

5. Witters, L. M., Curley, E. M., Kumar, R., Chinchilli, V. M., Harvey, J. P., Crebbin, V., Harvey, H. A., Lipton, A. Epidermal growth factor receptor ectodomain in the urine of patients with squamous cell carcinoma. Clinical Cancer Research, 1:551-557, 1995.

6. Partanen R, Hemminki K, Koshkinen H, J C Luo, W P Carney, P W Brandt-Rauf. The detection of increased amounts of the extracellular domain of the EGFR in serum during carcinogenesis in asbestosis patients. J. of Occupational Medicine: 36: 1324-1328, 1994.

7. Moscatello D K, Holgado-Madruga M, Godwin A K, Ramirez G, Gunn G, Zoltick P W, Biegel J A, Hayes R C, Wong A J. Frequent expression of a mutant EGF Receptor in multiple human tumors. Cancer Res. 55:5536-5539, 1995.

8. Moscatello, D. K., Ramirez, G., and Wong, A. A naturally occurring mutant human epidermal growth factor receptor as a target for peptide vaccine immunotherapy of tumors. Cancer Research, 57: 1419-1424, 1997.

9. Humphrey, P. A., Wong, A. J., Volgelstein, B., Zalutsky, M. R., Fuller, G. N., Archer, G., Friedman, H. S., Kwatra, M. M., Bigner, S. H., and Bigner, D. D. Antisynthetic peptide antibody reacting at the fusion junction of deletion-mutant epidermal growth factor receptors in human glioblastoma. Proceedings of the National Academy of Science, USA, 87: 4207-4211, 1990.

10. Garcia de Palazzo, I. E., Adams, G. P., Sundareshan, P., Wong, A. J., Testa, J. R., Bigner, D. D., and Weiner, L. M. Expression of mutated epidermal growth factor receptor by non-small cell lung carcinomas. Cancer Research, 53: 3217-3220, 1993.

11. Lorimer I A J, Keppler-Hafkemeyer A, Beers R A, Pegram C N, Bigner D D, Pastan I. Recombinant immunotoxins specific for a mutant epidermal growth factor receptor: targeting with a single chain antibody variable domain isolated by phage display. Proc. Natl. Acad. Sci. USA, 93: 14815-14820, 1996.

12. Nagane M, Levitski A, Gazit A, Cavenee W K, Huang H-J S. Drug resistance of human glioblastoma cells conferred by a tumor-specific mutant epidermal growth factor receptor through modulation of Bcl-XL and caspace-3-like proteases. Proc. Natl. Acad. Sci. USA 95: 5724, 1998.

13. Ali S M, Leitzel K, Lucas T, Chinchilli V, Engle L, Witters L, Sanders S, Demers L, Harvey H, Lipton A. HER-2/neu levels by ELISA in breast cancer homogenates. Proc. Amer. Soc. Clin. Oncol. 18: 617a, abstract # 2386, 1999.

We claim:

1. A method of detecting and quantifying EGFRvIII in a mammal, comprising performing an ELISA specific for EGFRvIII with a biological sample from said mammal.

2. The method of claim 1, wherein the biological sample is at least one of the group of urine, serum, plasma, CSF, amniotic fluid, breast secretions, lung sputum, or tumor cell extracts.

3. A method of detecting cancer in a mammal, comprising performing an ELISA specific for EGFRvIII with a biological sample from said mammal.

4. The method of claim 3, wherein the biological sample is at least one of the group of urine, serum, plasma, CSF, amniotic fluid, breast secretions, lung sputum, or tumor cell extracts.

5. The method of claim 3, wherein said cancer is at least one of the group of breast cancer, adenocarcinoma, squamous lung cancer, gastrointestinal cancer, renal cell cancer, bladder cancer, glioma, gynecological carcinoma, or prostate cancer.

6. A method of selecting a mammal with cancer for novel mutant EGF-directed anticancer therapies from at least one of the group of a vaccine, an antibody-toxin conjugate, or EGFRvIII-specific tyrosine kinase inhibitors, comprising performing an ELISA specific for EGFRvIII with a biological sample from said mammal, analyzing results of said EL:ISA, and selecting at least one of the group of said mutant EGF-directed anticancer therapies.

7. The method of claim 6, wherein the biological sample is at least one of the group of urine, serum, plasma, CSF, amniotic fluid, breast secretions, lung sputum, or tumor cell extracts.

8. An ELISA for the sensitive detection of wild type and/or EGFRvIII in a mammalian sample of urine, serum, plasma, CSF, amniotic fluid, breast secretions, lung sputum, tumor cell extracts, or any extracellular or cellular fluids.

9. A method of detecting a preneoplastic lesion in a mammal, comprising performing an ELISA specific for EGFRvIII with a biological sample from said mammal.

10. The method of claim 9, wherein the preneoplastic lesion is Barrett's esophagus.

11. A method of detecting benign prostatic hyperplasia in a mammal, comprising performing an ELISA specific for EGFRvIII with a biological sample from said mammal.

12. A method of generating antibodies specific for EGFRvIII, comprising:
preparation of an antibody against the mutant EGF receptor by immunizing a mammal with at least one of a mutant receptor protein, an epitope of said mutant receptor protein, a sequence that mimics said epitope, or DNA encoding said mutant receptor protein or epitope;
obtaining a high titer antibody preparation from said mammal, said antibody preparation recognizing mutant EGF and wild type (wt) receptor;
pooling bleeds from said mammal, concentrating and partially purifying said bleeds by precipitation;
obtaining a pellet from said precipitation and dialyzing said pellet; and
passing said (antibody preparation) dialyzed pellet over an affinity matrix column (with said epitope) and eluting antibodies from said column to obtain antibodies specific for EGFRvIII.

13. The method of claim 12, wherein said epitope comprises EKKGNYVV (SEQ ID NO:5), a fragment of said sequence, or a modification of said sequence.

14. The method of claim 12, wherein said epitope comprises LEEKKGNYVVTDH (SEQ ID NO:1), a fragment of said epitope, or a modification of said epitope.

15. The method of claim 12, wherein said epitope comprises KGN (SEQ ID NO:6) or a modification of said epitope.

16. The method of claim 12, wherein said epitope comprises LEEKKC (SEQ ID NO:2), a fragment of said epitope, or a modification of said epitope.

17. The method of claim 12, wherein said epitope comprises EKK (SEQ ID NO:7) or a modification of said epitope.

18. The method of claim 12, wherein said epitope comprises NYVVTDH (SEQ ID NO:8), a fragment of said epitope, or a modification of said epitope.

19. The method of claim 12, wherein said epitope comprises NYV (SEQ ID NO:9) or a modification of said epitope.

20. A method of generating antibodies specific for EGFRvIII, comprising:
preparation of an antibody against the mutant EGF receptor by immunizing a mammal with at least one of a mutant receptor protein, an epitope of said mutant receptor protein, a sequence that mimics said epitope, or DNA encoding said mutant receptor protein or epitope;
obtaining serum from said; and
passing said serum over an affinity matrix column and eluting antibodies from said column to obtain antibodies specific for EGFRvIII.
